(19) United States
(12) Patent Application Publication
(10) Pub. No.: US 2006/0153888 A1
(43) Pub. Date: Jul. 13, 2006

(54) Methods for scavenging oxidizing nitrogen and oxygen species with fragrances having antioxidative properties

(75) Inventors: Jesse C. Leverett, Rockford, MI (US); James R. Mayne, Lowell, MI (US); Stephen R. Missler, Grand Rapids, MI (US); John V. Scimeca, Kentwood, MI (US); Richard C. Roth, Ada, MI (US)

Correspondence Address: IN RE: ALTICOR INC. 28533;BRINKS, HOFER, GILSON & LIONE, P.O. BOX 10395, CHICAGO, IL, 60610, US

(73) Assignee: Access Business Group International LLC

(21) Appl. No.: 11/296,799

(22) Filed: Dec. 7, 2005

Related U.S. Application Data
(60) Provisional application No. 60/633,752, filed on Dec. 7, 2004.

(51) Int. Cl. A61K 36/899; A61K 8/97

(52) U.S. Cl. 424/401; 424/750

(57) ABSTRACT

Methods and compositions for scavenging reactive molecules, including free radicals, reactive oxygen and reactive nitrogen species, in the air are described. More specifically, methods and compositions for scavenging reactive molecules in the air before the reactive chemicals contact the skin or other tissue surface are described. The scavenging occurs by topical application of a scavenging agent, such as a volatile antioxidant, to the skin, or by releasing a scavenging agent, such as a volatile antioxidant, into the air near or surrounding the skin. Additionally, the present invention relates to compositions comprising a scavenging agent in combination with a carrier or vehicle, wherein the scavenging agent may include fragrances or fragrance components having antioxidant properties and wherein the scavenging agent scavenges reactive molecules in the air space surrounding a site on the skin where the composition has been applied. The compositions of the invention also may be used in household items such as candles, deodorizers and pet litter to scavenge reactive oxygen and nitrogen species in the home.

This application claims priority to U.S. Provisional Application Ser. No. 60/633,752, filed Dec. 7, 2004, the entire contents of which are incorporated by reference herein.

### FIELD OF THE INVENTION

The invention relates to methods and compositions for scavenging reactive molecules, including free radicals, reactive oxygen and reactive nitrogen species, in the air. More specifically, the present invention relates to methods and compositions for scavenging reactive molecules in the air before the reactive chemicals contact the skin, eyes, mucous membranes or other tissue surfaces. The scavenging occurs by topical application of a scavenging agent, such as a volatile antioxidant, to the skin, including the skin surrounding or near the eyes and mucous membranes, or by releasing a scavenging agent, such as a volatile antioxidant, into the air near or surrounding the skin, eyes, or mucous membranes. Additionally, the present invention relates to compositions comprising a scavenging agent in combination with a carrier or vehicle, wherein the scavenging agent may include fragrances or fragrance components having antioxidant properties and wherein the scavenging agent scavenges reactive molecules in the air space surrounding a site on the skin where the composition has been applied.

### BACKGROUND OF THE INVENTION

Free radicals and other oxidants have been implicated as contributing causes to skin conditions such as photo damage, general aging of the skin, contact dermatitis, wrinkling, inflammation and damage to the skin tissue. Free radicals and reactive oxygen and nitrogen species act directly, or as intermediates, on cell membranes to adversely impact the skin. Topical skin treatments may provide defense against reactive compounds that are detrimental to the maintenance of healthy skin.

### SUMMARY OF THE INVENTION

The present invention includes compositions for and methods of scavenging reactive molecules such as free radicals and other reactive oxygen or reactive nitrogen species by releasing a scavenging agent into a volume of air. The scavenging agent may include a fragrance or fragrance component or the scavenging agent may be a fragrance. The scavenging agent and/or the fragrance component comprises an aromatic ring and at least one free hydroxyl group attached directly to the ring. The scavenging agent and/or fragrance component may further comprise one or more additional electron donating groups such as an alkyl, ether, hydroxyl or amine group attached to the aromatic ring.

Further embodiments of the compositions and methods described herein require the step of releasing the scavenging agent into the volume of air by one or more of the exemplary actions: lighting a candle, activating a room deodorizer, scratching pet litter or by applying a fragrance, cosmetic or moisturizing product to a site of application on an area of skin whereby the scavenging agent enters an air space adjacent to the site of application and is available for the scavenging of free radicals and reactive oxygen or nitrogen species. Of course, other methods of releasing a scavenging agent in the air also are envisioned.

Thus, in one embodiment, the invention comprises compositions and a method of scavenging free radicals and reactive oxygen or reactive nitrogen species by releasing a scavenging agent and/or a fragrance component into a volume of air, wherein the scavenging agent and/or the fragrance component comprises at least one of vanillin, vanillyl alcohol, vanillal bourbonal (or ethyl vanillin), raspberry ketone, eugenol, ferulic acid, orange terpenes, lilial, lemon grass oil, lemon grass, jasmopyrane, paracymene, florosa, balsamic compounds (e.g. anise, balsam, caramel, chocolate, cinnamon, honey), resorcinol, catechol, parabens (e.g. ethyl paraben), thymol, maltol, 2-phenoxyethanol, 3-phenyl-1-propanol, coumarin, limonene, geraniol, camphor, menthol, ethyl monanoate, butylated hydroxytoluene, Vetiver Haiti, Betiver Java, American clary sage, clary sage, clove bud, clove leaf, sandalwood oil, Australian myrtle oil, or combinations thereof.

In another embodiment, the invention comprises a method for preventing damage to skin tissue comprising selecting a scavenging agent that is volatile at temperatures of at least 0° C. In another embodiment, the scavenging agent is volatile at temperatures ranging from about 5-45° C. In a further embodiment, the scavenging agent is volatile at temperatures ranging from about 20-35° C. Therefore, one embodiment of the invention is a method of mixing a volatile scavenging agent with a vehicle, such as a fragrance vehicle, and applying the mixed agent and vehicle to an area of skin, whereby the scavenging agent enters an air space adjacent to the area of skin and scavenges free radicals, reactive oxygen or reactive nitrogen species in the air space.

Still another embodiment of the invention is a composition comprising a free radical scavenging agent and a vehicle, such as a fragrance vehicle, whereby the free radical scavenging agent enters an air space adjacent to a site of application and is available for the scavenging of free radicals, reactive oxygen or reactive nitrogen species.

### BRIEF DESCRIPTION OF THE FIGURES

FIG. 1 depicts a plate map for a NORA Assay.

FIG. 2 is a graph of a gas chromatography analysis with flame ionization detector (GC-FID) of butylated hydroxytoluene (BHT).

FIG. 3 is a graph of a GC-FID analysis of cinnamyl alcohol.

FIG. 4 is a graph of a GC-FID analysis of ferulic acid.

FIG. 5 is a graph of a GC-FID analysis of eugenol.

FIG. 6 is a graph of a GC-FID analysis of geraniol.

FIG. 7 is a graph of a GC-FID analysis of thymol.

FIG. 8 is a graph of a GC-FID analysis of Vetiver Java.

FIG. 9 is a graph of a GC-FID analysis of Lemongrass Oil.

FIG. 10 is a graph of a GC-FID analysis of Clove bud.

FIG. 11 is a graph of a GC-FID analysis of myrtle.

### DETAILED DESCRIPTION OF THE INVENTION

It is to be understood that this invention is not limited to the particular methodology, protocols, or compositions described herein. It is also to be understood that the terminology used herein is for the purpose of describing particular embodiments only, and is not intended to limit the scope of the present invention.

Unless defined otherwise, all technical and scientific terms used herein have the same meaning as commonly understood to one of ordinary skill in the art to which this invention belongs. The following definitions are provided in order to aid the reader in understanding the detailed description of the present invention, as well as to aid in the understanding of the appended claims.

As used herein, the singular forms “a,” “an,” and “the” include plural reference unless the context clearly dictates otherwise. Thus, for example, reference to “a free radical scavenging agent” is a reference to one or more free radical scavenging agents and includes equivalents thereof known to those skilled in the art, and so forth.

The term “antioxidant” means a substance that prevents or slows the rate of oxidation reactions.

The terms “fragrance” or “fragrance component” mean a material or combination of materials used to produce an odor or used to mask an odor. When used to mask an odor, fragrances or fragrance components may or may not have a detectable odor of their own. A fragrance or fragrance component may be or may be included with a scavenging agent.

The term “scavenging agent” refers to any molecule or compound capable of removing or eliminating a reactive component from a chemical reaction. In one embodiment, the scavenging agent is a volatile antioxidant. In another embodiment, the scavenging agent is a non-volatile antioxidant. In a further embodiment, the scavenging agent is a combination of at least one volatile and at least one non-volatile antioxidant. In another embodiment, the scavenging agent comprises a molecule having one or more of the following characteristics: an aromatic ring; at least one free hydroxyl group attached directly to the ring; absence of electron withdrawing groups attached to the ring (such as carboxylic acid/ester moieties); and presence of one or more electron donating groups such as alkyl, ether, hydroxyl, or amine groups attached directly to the ring. In another embodiment, the scavenging agent has one or more of the following characteristics: unsaturated hydrocarbons or hydrocarbons containing at least one functional group selected from the group consisting of: carbonyl, amine, diamine, cyclic acetyl and enol ether. Therefore, a scavenging agent may have, for example an aromatic ring and at least one free hydroxyl group attached directly to the ring. A scavenging agent may be or may include a fragrance or fragrance component.

The term “oxidation” means the loss of at least one electron by a compound or element.

The term “reactive” is used to refer to elements or compounds that are available to react chemically.

The term “reduction” means the gain of at least one electron by a compound or element.

The term “scavenge” means to remove or eliminate a reactive component from a chemical reaction.

The term “volatile” means a substance that changes easily from a solid or liquid to vapor or gas state.

The present invention is based on the surprising discovery that volatile antioxidant compounds may be administered to the skin, eyes, or mucous membranes or to the air surrounding, near, or adjacent to the skin, eyes, or mucous membranes and thereby protect skin tissue, eye tissue, and mucous membranes from damage due to exposure to reactive molecules such as free radicals and reactive oxygen and nitrogen species. For example, the volatile antioxidant compounds may be administered in cosmetic or fragrance compositions and in such a form, the volatile antioxidant compounds may be used to scavenge free radicals from the atmosphere around the body and, more specifically, from the air adjacent to the site of application. Prior to this discovery, non-volatile antioxidants were used to protect the skin, eyes and mucous membranes from damaging scavenging free radicals, and reactive oxygen and nitrogen species. These non-volatile antioxidant compounds may provide some protection to the skin, eyes and mucous membranes but use of non-volatile antioxidant compounds allows the oxidizer (e.g. the free radical, reactive oxygen, or reactive nitrogen species) to contact the skin, eye or mucous membrane and potentially cause damage prior to scavenging. Therefore, the current state of the art does not recognize the unique ability of volatile antioxidant compounds to prevent or reduce skin and tissue damage by preventing or reducing a number of reactive chemicals reaching a skin or tissue surface.

Thus, in one embodiment, the invention comprises a scavenging agent that combines at least one volatile and at least one non-volatile antioxidant compound. In another embodiment, the invention consists essentially of volatile scavenging agents. In a further embodiment, the invention consists of volatile scavenging agents.

The methods and compositions described herein are not limited to application in personal cosmetic, such as moisturizers, shampoos, gels, etc. and fragrances, but also are applicable to use of scavenging agents in the formulation of household items (such as candles, pet litter and room deodorizers).

The compositions of the present invention may be formulated as a solution, gel, lotion, cream, ointment, oil-in-water emulsion, water-in-oil emulsion, stick, spray, paste, mousse, tonic, or other suitable form.

One of ordinary skill in the art will appreciate that many different compounds may be used in practicing the present invention. For example, the compositions and methods of the present invention may include any of vanillin, vanillyl alcohol, vanillal bourbonal (or ethyl vanillin), raspberry ketone, eugenol, ferulic acid, orange terpenes, lilial, lemon grass oil, lemon grass, jasmopyrane, paracymene, florosa, balsamic compounds (e.g. anise, balsam, caramel, chocolate, cinnamon, honey), resorcinol, catechol, parabens (e.g. ethyl paraben), thymol, maltol, 2-phenoxyethanol, 3-phenyl-1-propanol, coumarin, limonene, geraniol, camphor, menthol, ethyl monanoate, butylated hydroxytoluene, Vetiver Haiti, Betiver Java, American clary sage, clary sage, clove bud, clove leaf, sandalwood oil, Australian myrtle oil, or combinations thereof.

In one example, a composition of the present invention may be used to prevent or reduce skin or tissue damage due to nitric oxide free radicals. A composition for preventing or reducing skin or tissue damage caused by nitric oxide free radicals may comprise a scavenging agent having one or more of the following characteristics:
1. An aromatic ring;
2. At least one free hydroxyl group attached directly to the ring;
3. Absence of electron withdrawing groups attached to the ring (such as carboxylic acid/ester moieties);
4. Presence of one or more electron donating groups such as alkyl, ether, hydroxyl or amine groups attached directly to the ring;
5. At least one unsaturated hydrocarbon; and
6. Hydrocarbons containing at least one functional group selected from the group consisting of: carbonyl, amine, diamine, cyclic acetyl, and enol ether.

These characteristics provide a stable molecule with rich electron densities that can rapidly scavenge oxidizing species such as nitric oxide free radicals and ozone.

In one embodiment of the invention, a composition of the invention may include or a method of the invention may utilize scavenging agents that are fragrant oils having antioxidative properties. Table 1, found below, provides a measure of the antioxidant activity of a test compound against free radicals (DPPH *AO Power value) as well as a measure of the antioxidant activity of a test compound against nitric oxide free radicals (NORA AP Power value).

In one embodiment of the invention, a composition of the invention may include or a method of the invention may utilize scavenging agents that are fragrant oils having antioxidative properties. Table 1, found below, provides a measure of the antioxidant activity of a test compound against free radicals (DPPH *AO Power value) as well as a measure of the antioxidant activity of a test compound against nitric oxide free radicals (NORA AP Power value).

TABLE 1
Antioxidative Properties of Fragrant Oils:

| | | | NORA |
|---|---|---|---|
| Test Compound | Manufacturer | DPPH *AO Power | AO Power |
| Vetiver Haiti | Lebermuth | <0.0035 | 123 |
| Vetiver Java | Lebermuth | <0.0035 | 192 |
| American Clary | Lebermuth | <0.0035 | 32 |
| Sage | | | |
| Clary Sage | Lebermuth | <0.0035 | 34 |
| Lemongrass | Lebermuth | <0.0035 | 126 |
| Clove bud | Lebermuth | <0.0035 | 94 |
| Clove leaf | Lebermuth | <0.0035 | 29 |
| Vanillin | Berje | 0.0112 | 93 |
| Ethyl Vanillin | Berje | 0.0135 | 96 |
| BOIS II | Barnet | <0.0035 | 1 |
| Sandalwood | Presperse | <0.0035 | 61 |
| Oil | | | |
| Australian | Southern Cross | 0.00426 | 41 |
| Myrtle Oil | | | |

*AO Power: Antioxidant Power equals 1/EC₅₀, where EC₅₀ is the efficient concentration of concentration required to inhibit oxidation by 50%. Expressed in units/gram.

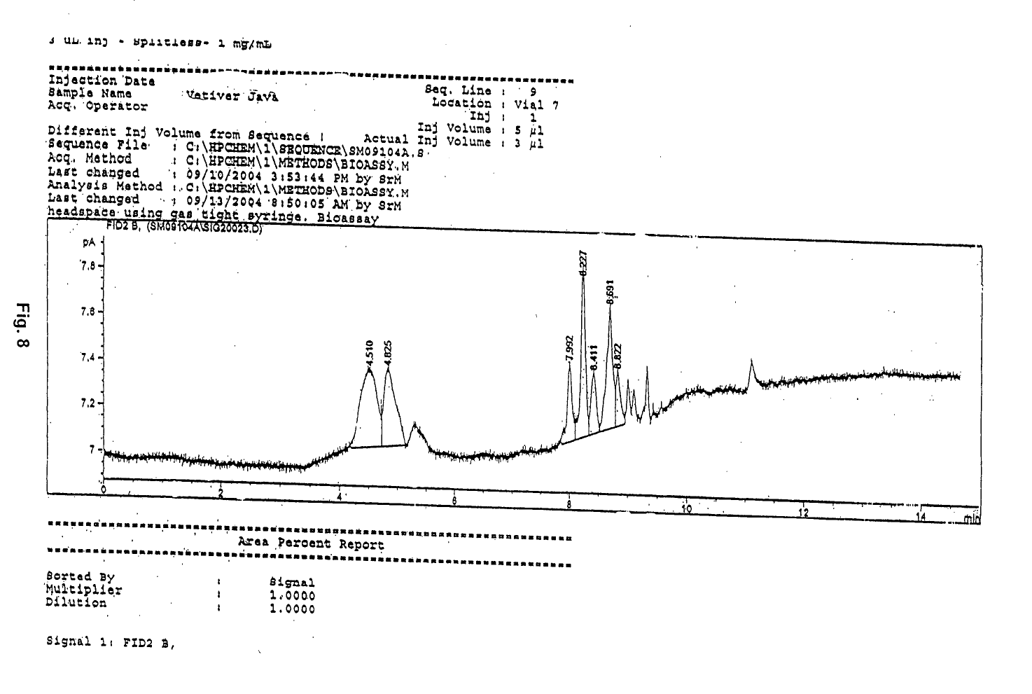

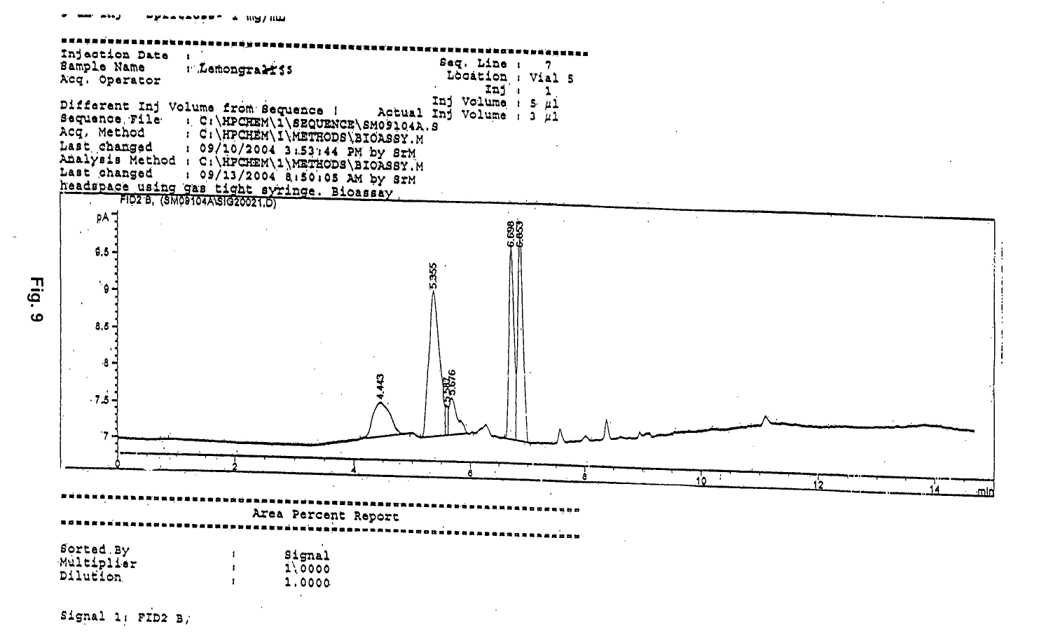

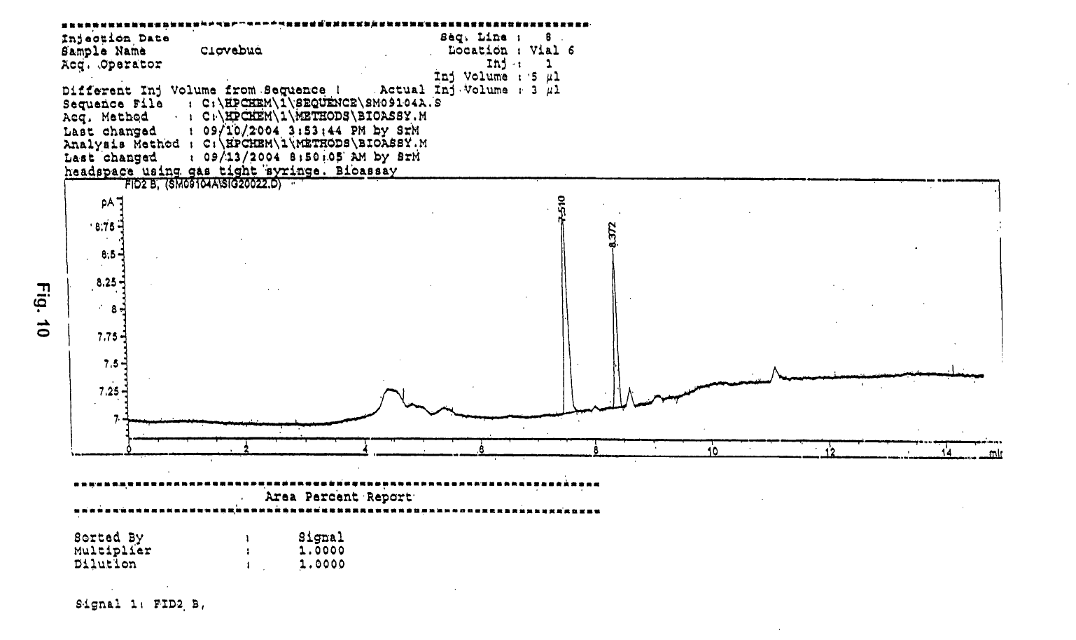

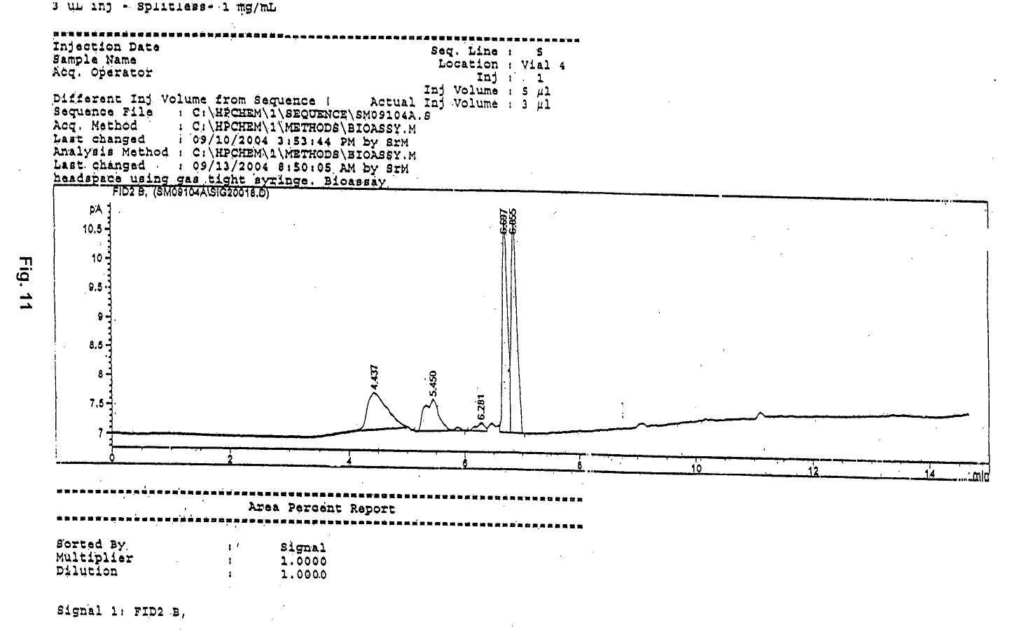

The oils listed above in Table 1 have volatile components as depicted in FIGS. 8-11. Specifically, FIGS. 8-11 provide measures of the volatility of various compounds as depicted in the graphs from the gas chromatography analysis with flame ionization detector (GC-FID). FIGS. 8-11 demonstrate the volatility of compounds present in Vetiver Java, Lemongrass Oil, Clove bud and myrtle. FIGS. 8-11 illustrate that each of these oils have two or more volatile components.

Therefore, in one embodiment the scavenging agent of the present invention comprises a volatile component. In another embodiment, the scavenging agent comprises at least one volatile and at least one non-volatile component. Indeed, the present invention is based on the surprising discovery that scavenging agents having a volatile component provide additional protection against reactive molecules, such as free radicals, reactive nitrogen and reactive oxygen species. In particular, the volatile component can volatize and react with reactive molecules in the air above, near, surrounding or adjacent to a tissue and thereby prevent the reactive molecule from reaching the tissue and causing damage. Alternatively, a volatile scavenging agent may neutralize a reactive molecule before it reaches the skin or tissue surface, thereby preventing or reducing tissue damage caused by reactive molecules.

In one embodiment, the scavenging agent comprises a volatile component and the scavenging agent is topically administered to the skin. In a further embodiment, the volatile component in the scavenging agent topically applied to the skin may volatize at body temperature, e.g. 37° C. In yet a further embodiment, the scavenging agent may be volatile at temperatures ranging from 5-45 ° C. In an alternative embodiment, the scavenging agent may be volatile at a temperature in the range of 20-35 ° C.

One of ordinary skill in the art will appreciate that the scavenging agent of the present invention may be combined with a carrier or vehicle. The carrier or vehicle may act as a diluent, dispersant, or carrier for other materials present in the composition, so as to facilitate their distribution when the composition is applied to the skin. One of ordinary skill in the art also will appreciate that vehicles other than water can include liquid or solid emollients, solvents, humectants, thickeners, and powders. In general, suitable vehicles according to the present invention may comprise, but are not limited to comprising, any of the following examples: water; castor oil; ethylene glycol monobutyl ether; diethylene glycol monoethyl ether; corn oil; dimethyl sulfoxide; ethylene glycol; isopropanol; soybean oil; glycerin; soluble collagen; zinc oxide; titanium oxide; Kaolin; or hyaluronic acid.

Additionally, suitable vehicles used in the present invention may optionally comprise one or more humectants, including but not limited to: dibutyl phthalate; gelatin; glycerin; soluble collagen; sorbitol; or sodium 2-pyrrolidone-5-carboxylate. Other examples of humectants that may be used in practicing the present invention are found at page 575 of the CFTA Cosmetic Ingredient Dictionary and Handbook (10th ed.), and are incorporated herein by reference.

Additionally, suitable vehicles in the present invention may optionally comprise one or more emollients including but not limited to: stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate. Other examples of emollients that may be used in practicing the present invention are found at pages 572-575 of the CFTA Cosmetic Ingredient Dictionary and Handbook (10th ed.), and are incorporated herein by reference.

As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials.

In one example, a particularly convenient form of the composition is an emulsion, in which case an oil or oily material (emollient) will normally be present, together with an emulsifier to provide either a water-in-oil emulsion or an oil-in-water emulsion.

The composition can also comprise water, usually up to 95%, preferably from 5 to 95% by weight.

The composition can also optionally comprise a high molecular weight silicone surfactant that can also act as an emulsifier, in place of or in addition to the optional emulsifier(s) already mentioned.

The silicone surfactant may be a high molecular weight polymer of dimethyl polysiloxane with polyoxethylene and/or polyoxpropylene side chains having a molecular weight of from 10,000 to 50,000. When used, the dimethyl polysiloxane polymer is conveniently provided as a dispersion in a volatile siloxane, the dispersion comprising, for example, from 1 to 20% by volume of the polymer and from 80 to 99% by volume of the volatile siloxane. Ideally, the dispersion consists of 10% by volume of the polymer dispersed in the volatile siloxane.

Examples of the volatile siloxanes in which the polysiloxane polymer can be dispersed include polydimethyl siloxane (pentamer and/or hexamer).

A preferred silicone surfactant is cyclomethicone and dimethicone copolyol, such as DC 3225C Formulation Aid available from DOW CORNING. Another is laurylmethicone copolyol, such as DC Q2-5200, also available from Dow Corning.

The amount of silicone surfactant, when present in the composition will normally be up to 25%, preferably from 0.5 to 15% by weight of the emulsion.

Other suitable vehicles and compositions that may be used in practicing the present invention will be apparent to those of skill in the art and are included within the scope of the present invention.

The compositions of the present invention may also contain various known and conventional cosmetic adjuvants so long as they do not detrimentally affect the desired scavenging or antioxidant activity provided by the composition. For example, the composition of the present invention can further include one or more additives or other optional ingredients well known in the art, which can include but are not limited to preservatives, such as para-hydroxy benzoate esters; antioxidants, such butyl hydroxytoluene; humectants, such as glycerol, ethoxylated glycerins such as glycereth-26, sorbitol, 2-pyrrolidone-5-carboxylate, dibutylphthalate, gelatin, polyethylene glycol, such as PEG 200-600; buffers together with a base such as triethanolamine or sodium hydroxide; waxes, such as beeswax, ozokerite wax, paraffin wax; plant extracts, such as Aloe Vera, cornflower, witch hazel, elderflower, cucumber; as well as acerola cherry fermentate, thickeners; activity enhancers; colorants; and perfumes. Cosmetic adjuvants can form the balance of the composition. Other suitable additives and/or adjuvants are described in U.S. Pat. No. 6,184,247, the entire contents of which are incorporated herein by reference.

It may also be desirable to incorporate anti-inflammatory and/or anti-irritant agents. The natural anti-inflammatory and/or anti-irritant agents are preferred. For example, licorice and its extracts, dipotassium glycyrrhizinate, oat and oat extracts, candelilla wax, alpha bisabolol, aloe vera, Manjistha (extracted from plants in the genus Rubia, particularly Rubia cordifoliaI), and Guggal (extracted from plants in the genus Commiphora, particularly Commiphora Mukul), may be used. Skin conditioning agents such hyaluronic acid, its derivatives and salts including sodium hyaluronate, plant extracts such as kola nut, guarana mate, algae extract and skin benefit agents such as ceramides, glycoceramides, pseudoceramides, sphingolipids such as sphingomyelins, cerebrosides, sulphatides, and ganglioside, sphingosines, dihydrosphingosine, phytosphingosines, phospholipids, may also be incorporated, either separately or in mixtures. Fatty acids may also be combined with these skin benefit agents. For example, the ceramides and glycoceramides include those described in U.S. Pat. Nos. 5,589,178, 5,661,118, and 5,688,752, the relevant portions of which are incorporated herein by reference. For example, the pseudoceramides include those described in U.S. Pat. Nos. 5,198,210; 5,206,020; and 5,415,855, the relevant disclosures of which are incorporated herein by reference.

Thus, in practicing the present invention, a cosmetic adjuvant may be a filler, (e.g., solid, semi-solid, liquid, etc.); carrier; diluent; thickening agent; gelling agent; vitamin, retinoid, and retinol (e.g., vitamin B3, vitamin A, etc.); pigment; fragrance; sunscreen or sunblock.

For example, a composition of the present invention can optionally comprise sunscreens such as inorganic and organic sunscreens to provide protection from the harmful effects of excessive exposure to sunlight during use of the composition of the present invention. Examples of suitable sunscreens include those described in the U.S. OTC Sunscreen Monograph, the contents of which is incorporated herein by reference. When a composition of the present invention includes a sunscreen component or is incorporated into a sunscreen, the composition of the invention may comprise from about 0.1 to about 10%, preferably from about 1 to about 5% by weight of an organic sunscreen material.

The composition optionally can also comprise as a sunscreen titanium dioxide or zinc oxide having an average particle size of from 1 to 300 nm, iron oxide having an average particle size of from 1 to 300 nm, silica, such as fumed silica having an average particle size of from 1 to 100 nm. It should be noted that silica, when used as an ingredient in the emulsion according to the invention can provide protection from infrared radiation.

Ultrafine titanium dioxide in either of two forms, namely water-dispersible titanium dioxide and oil-dispersible titanium dioxide also may be used in a composition of the present invention. Water-dispersible titanium dioxide is ultrafine titanium dioxide, the particles of which are uncoated or which are coated with a material to impart a hydrophilic surface property to the particles. Examples of such materials include aluminum oxide and aluminum silicate. Oil-dispersible titanium dioxide is ultrafine titanium dioxide, the particles of which exhibit a hydrophobic surface property, and which, for this purpose, can be coated with metal soaps such as aluminum stearate, aluminum laurate, or zinc stearate, or with organosilicone compounds.

By “ultrafine titanium dioxide” is meant particles of titanium dioxide having an average particle size of less than 100 nm, preferably from 10 to 40 nm and most preferably from 15 to 25 nm. The total amount of titanium dioxide that can optionally be incorporated in the composition according to the invention is from 1 to 25%, preferably from 2 to 10% and ideally from 3 to 7% by weight of the composition.

The compositions of the present invention may also include propellants such as propane, isobutane, dimethyl ether, carbon dioxide, nitrous oxide; and solvents such as ethyl alcohol, isopropanol, acetone, ethylene glycol monomethyl ether, diethylene glycol monobutyl ether, diethylene glycol monoethyl ether, or powders such as chalk, talc, fullers earth, kaolin, starch, gums, collodial silica, sodium polyacrylate, tetra alkyl and/or trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate.

To prepare the compositions according to the present invention, at least one scavenging agent is mixed with a vehicle. The scavenging agent may be present in an amount ranging from about 0.01% to about 20% by weight of the total composition. At least one scavenging agent may also be present in an amount from about 0.05% to about 15% by weight of the total composition. Alternatively, at least one scavenging agent may be present in an amount from about 1% to about 12% by weight of the total composition. At least one scavenging agent may also be present in an amount from about 2% to about 10% by weight of the total composition. In another alternative embodiment, at least one scavenging agent is present in an amount from about 3% to about 8% by weight of the total composition. In yet a further embodiment, at least one scavenging agent is present in an amount from about 4 to about 6% by weight of the total composition.

Generally, a composition of the present invention may be topically applied, sprayed, or otherwise released into the air surrounding a skin or tissue surface, at least on a daily basis for a period of time sufficient to bring about a decrease in skin or tissue damage due to free radicals, and reactive nitrogen and oxygen species. Application or aeration of a composition of the present invention may continue for any suitable period of time. More specifically, within a few days to within a few months of the initial application or aeration, a user may notice an improvement in skin and tissue texture and smoothness. It should be appreciated that the frequency with which the composition disclosed herein should be applied will vary depending on the desired level of prevention or reduction in damage. In particular, the degree of cosmetic enhancement will vary directly with the total amount of composition used and the frequency of use.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as” or “for example”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise.

It is intended that the foregoing detailed description be regarded as illustrative rather than limiting. The present invention is further illustrated by the following experimental investigations and examples, which should not be construed as limiting.

### EXAMPLES

### Example 1

### DPPH Spectrophotometer Assay Procedure

The free radical 2,2-Diphenyl-1-picrylhydrazyl (DPPH) may be used to determine free radical scavenging ability of test materials such as a scavenging agent of the present invention. The amounts of various scavenging agents required to consume 50% of the starting DPPH material (EC₅₀) are reported below in Table 2. Measurements are taken after the reaction between the test scavenging agent and DPPH reaches steady state. DPPH is a free radical that changes color when it reacts with an antioxidant. The color change in DPPH is an indication of antioxidant power. Antioxidant power is defined as 1/EC50, where EC50 is the concentration of antioxidant required to reduce the color of DPPH by 50%. Thus, DPPH serves as a model for free radical oxidation. Provided below are details for conducting a DPPH Spectrophotometer Assay:

Scavenging Agent Test Samples:

Weigh 100 mg sample into 15 ml conical centrifuge tube.

Add 1 ml DMSO. Vortex to mix.

Dilute samples in MeOH according to Table 2.

Dilute 1 mM DPPH (prepared in 80% MeOH) to 100 uM in 80% MeOH.

In 3 ml cuvette, add 200 uL sample dilution. Add 1.8 ml of 100 uM DPPH.

Mix well. Incubate 15 min at Room Temp. Read at 520 nM.

TABLE 2
Dilution scheme for DPPH Spectrophotometer assay

| Mg/ml Sample | Sample/dilution | uL diluent | |
|---|---|---|---|
| 10 | 100 mg sample | 1 | mL |
| 5 | 500 uL 10 mg/ml | 500 | uL |
| 2.5 | 500 uL 5 mg/ml | 500 | uL |
| 1 | 400 uL 2.5 mg/ml | 600 | uL |
| 0 | 0 uL | 1 | ml |

Alternative Dilution Scheme:

If cuvette appears cloudy due to precipitation of sample, prepare sample dilutions (Table 2) in DMF (or suitable solvent) instead of DMSO. Also prepare DPPH working solution (100 uM) in DMF (or suitable solvent) instead of MeOH.

### Example 2

### Nitric Oxide Radical Absorbance (NORA) Analysis

The NORA assay determines the ability of a compound to quench nitric oxide free radicals (NO*) produced by a NO* generator. In the assay, a target compound is oxidized using the nitric oxide generator NOR-3, resulting in increased fluorescence. The concentration required to inhibit the formation of fluorescence by 50% over the time frame of an analysis is reported as the EC₅₀. Provided below are details for conducting a Nitric Oxide Radical Absorbance (NORA) Assay:

Reagents:

Dihydrorhodamine 123, Molecular Probes (Cat# D632)

NOR-3 ((±)-(E)-Ethyl-2-[(E)-hydroxyimino]-5-nitro-3-hexeneamide),

Calbiochem (#489530)

Phosphate Buffer (Certified pH 7.00), Fisher (Cat# SB108-1)

N,N Dimethyl Formamide, Fisher (Cat# D119)

Dimethyl Sulfoxide, Fisher (Cat# D136)

Methanol, HPLC grade.

Reagent Prep:

DHR123: 1.0 mg/3 ml MeOH=1 mM stock (Freeze and protect from light) 7.5 uL stock/10 ml Buffer=0.75 uM working solution.

NOR-3: 1.0 mg NOR-3/100 uL DMSO. Vortex.
Bring to 10 ml with buffer=465 uM working solution.

Sample Prep:

Oil: Weigh 100 mg into 15 ml polypropylene centrifuge tube. Add 5 ml

DMF. Vortex to mix/

Prepare sample dilution in a 96-well polypropylene storage plate (2 mL/well), the dilution plate:
A. 60 uL Stock+1.94 ml DMF=200 ug/ml
B. 500 uL 200 ug/ml+500 ul DMF=100 ug/ml
C. 500 uL 100 ug/ml+500 ul DMF=50 ug/ml
D. 500 uL 50 ug/ml+500 ul DMF=25 ug/ml
E. 400 uL 25 ug/ml+600 ul DMF=10 ug/ml
F. 500 uL 10 ug/ml+500 ul DMF=5 ug/ml
G. 0 uL Sample+1000 ul DMF=blank
H. Positive Control

Positive Control: (1 uM Uric Acid)

Add 17 mg Uric Acid to 10 mL Alkaline Water (pH>12.0). Vortex and sonicate to dissolve=10 mM

Dilute 15 ul of 10 mM solution to 5 ml=30 uM

Dilute 100 uL of 30 uM solution to 1 ml. Add to row H of above dilution plate=3 uM.

Assay Procedure:

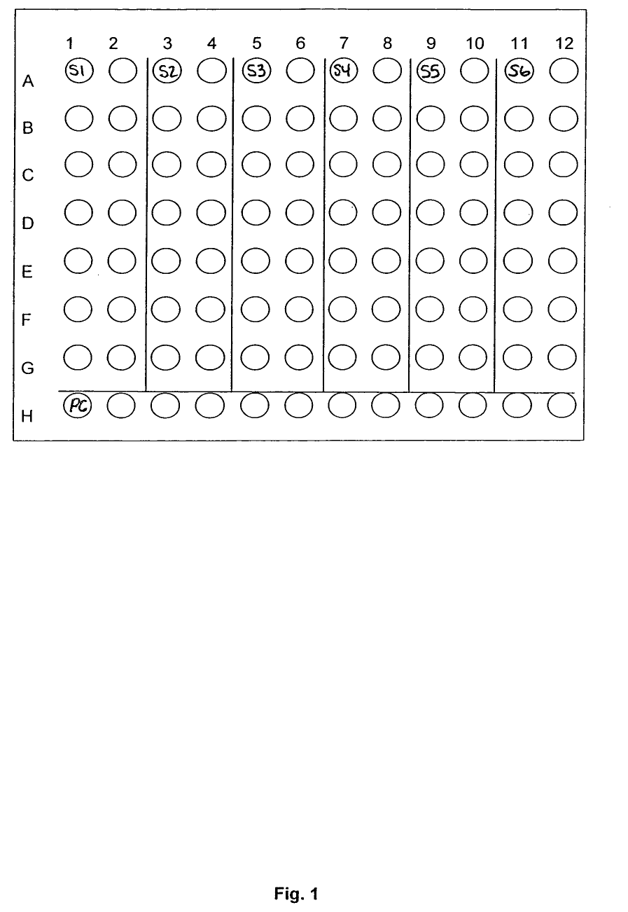

To 96 well black round bottom fluorescence assay plate: (as seen in FIG. 1)

Add 100 uL of each sample/PC/Blank in duplicate. Row H will contain PC only.

Add 100 ul DHR 123 to all wells

Add 70 uL Phosphate Buffer to all wells.

Add 30 uL NOR-3 to all wells.

Incubate plate 15 min at room temperature. Read at 485/535 nm.

### Example 3

### Volatile Compound Screening Assay

Volatility of compounds may be determined by capillary gas chromatography with a flame ionization detector (GC-FID) according to the procedure described below:

1. Place approximately 1 mg of scavenging agent test compound or sample in a 1.5 mL glass autosampler vial fitted with a screw cap having a teflon lined septum. Tightly close the cap and allow the sample to equilibrate at room temperature for 2 hours. Use water as a blank control.

2. Place sample vials in the autosampler of a gas chromatograph (GC) equipped for capillary columns using splitless injection, and having a flame ionization detector (FID). Set up GC conditions according to the GC method given below:

GC: HP6890 or equivalent

Column: DB-1 (J&W Scientific having dimensions of 25 m×0.32 mm with 25 um film thickness)

Carrier gas: Helium at 35 cm/sec linear velocity (˜12.4 psi, 1.5 mL/min)

Inlet conditions:

| | |
|---|---|
| Injection: | Splitless |
| Purge time: | 30 sec |
| Temperature: | 250 C. |
| Septum Sweep: | 2 mL/min |
| Total flow: | 44 mL/sec |

Detector conditions:

Detector: FID

| | |
|---|---|
| Temperature | 250 C. |
| Hydrogen Flow: | 40 mL/min |
| Air Flow: | 450 mL/min |
| Makeup flow: | 45 mL/min |
| Makeup gas: | Helium |

Oven Program:

| | |
|---|---|
| Initial temp.: | 75 C. |
| Initial time: | 1.00 min |
| Ramp 1: | 25 C./min |
| Final temp 1: | 230 C. |
| Final time 1: | 10 min |
| Run time: | 17.2 min |

Autosampler:

| Wash solvent: | Water |
|---|---|
| Sample washes: | 2 |
| Sample pumps: | 3 |
| Injection volume: | 3 uL |
| Syringe: | 10 uL gas tight |
| Post inj washes: | 5 |

Note:
Ensure syringe does not contact liquid/solid sample during injection.

1. Inject samples. Identify component peaks that do not appear in the blank. Peaks appearing above background are considered to be “volatile compounds”.

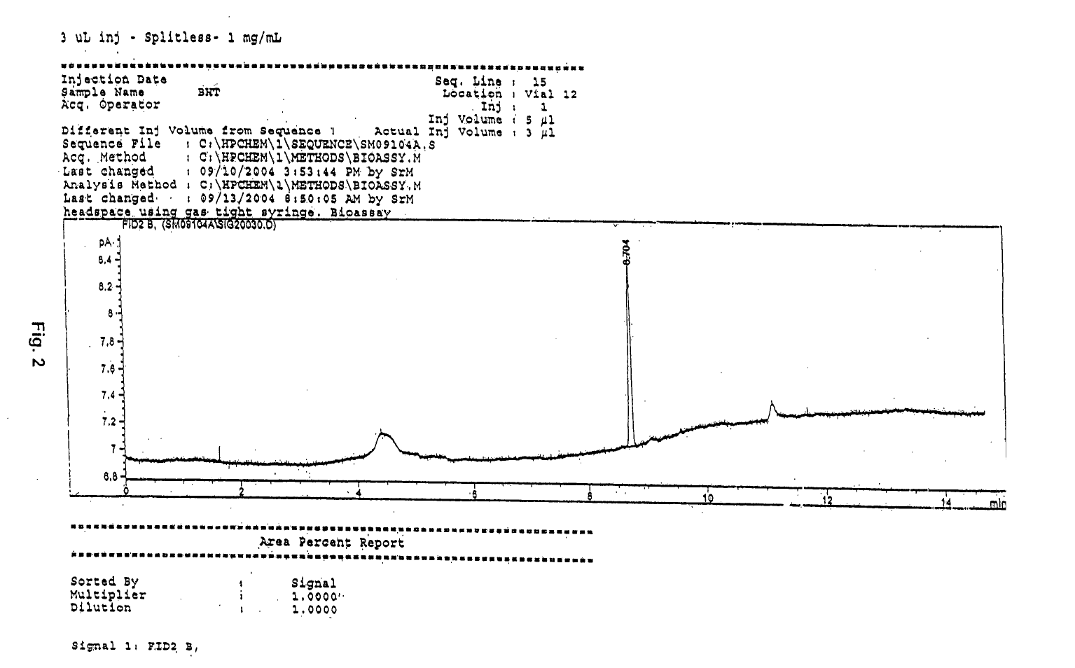

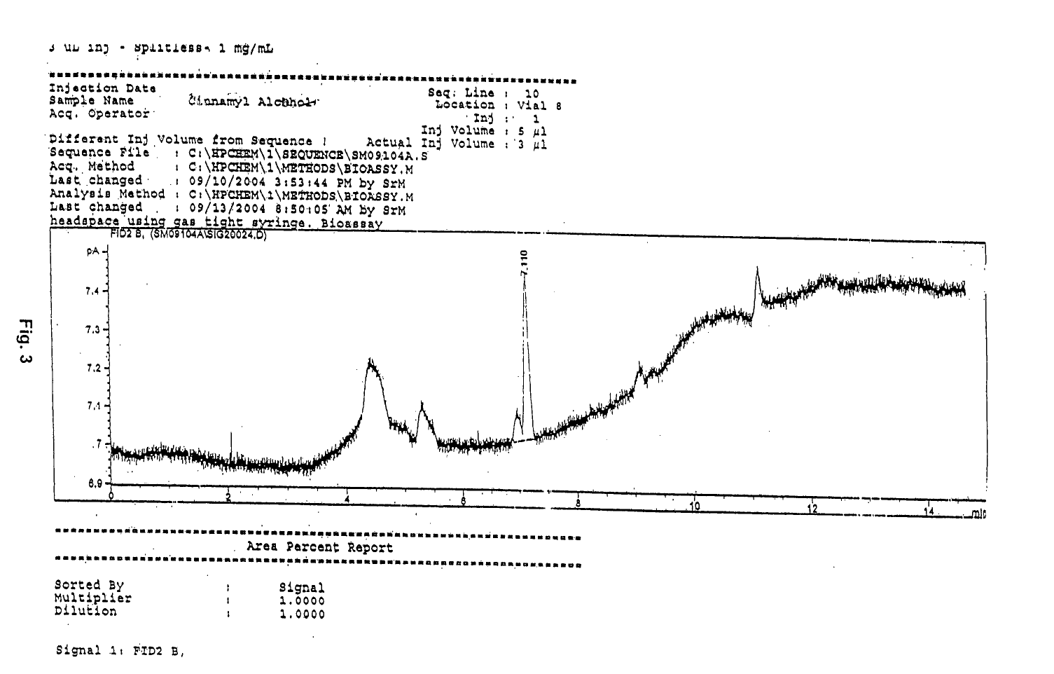

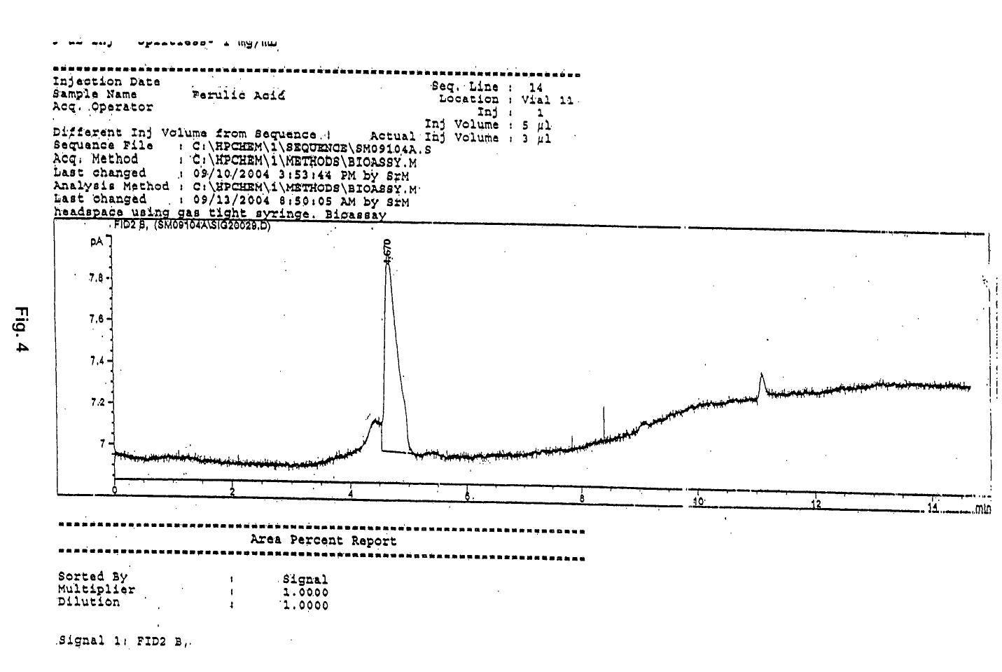

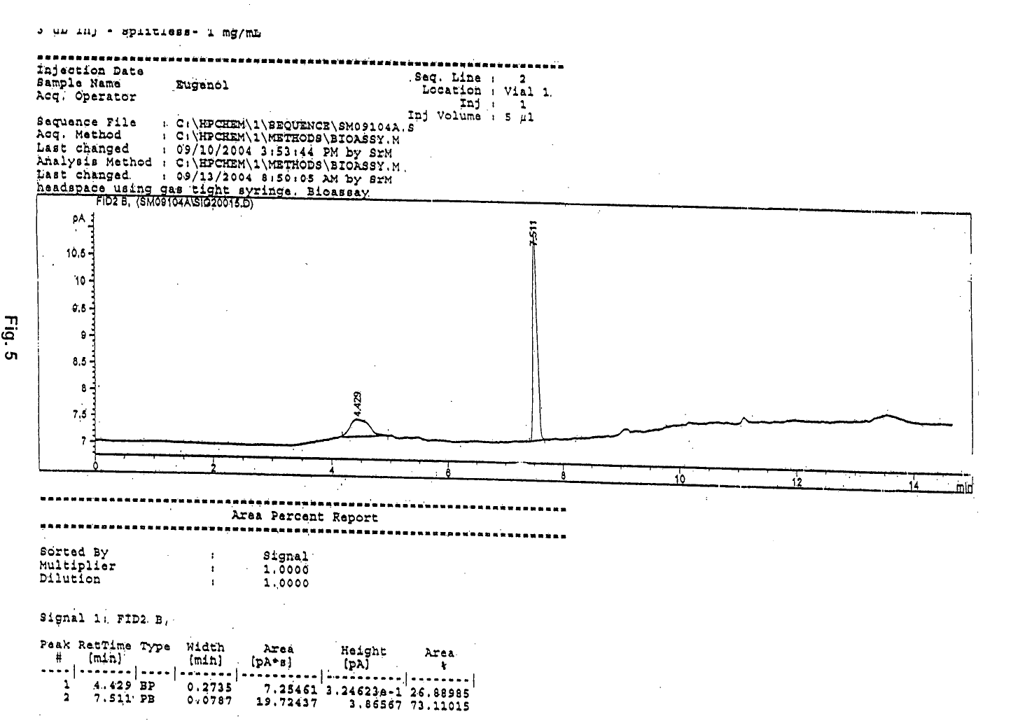

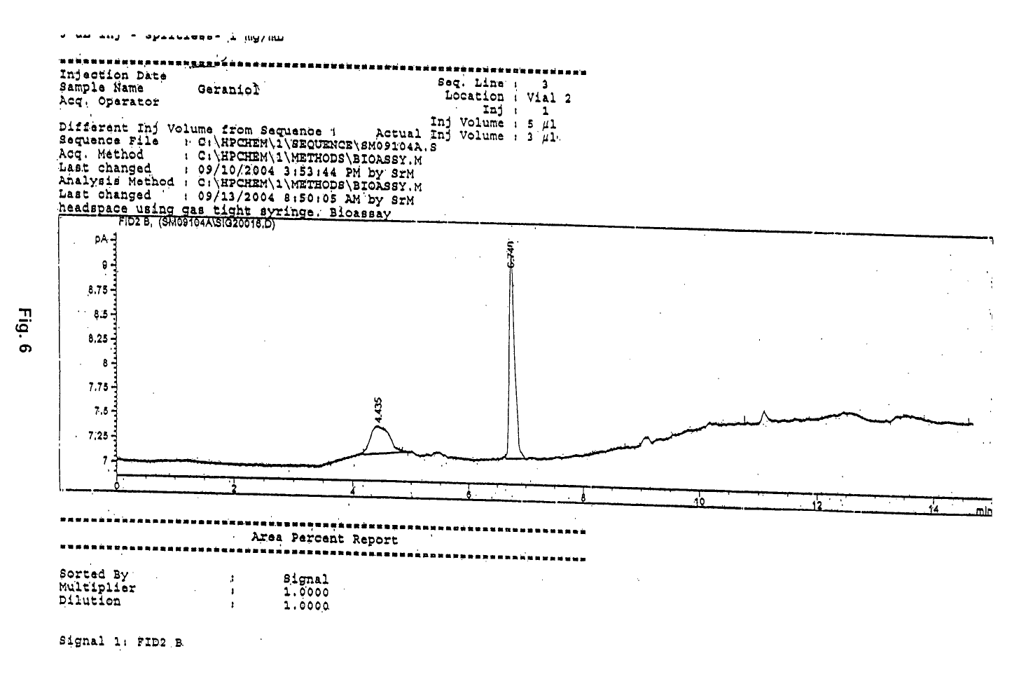

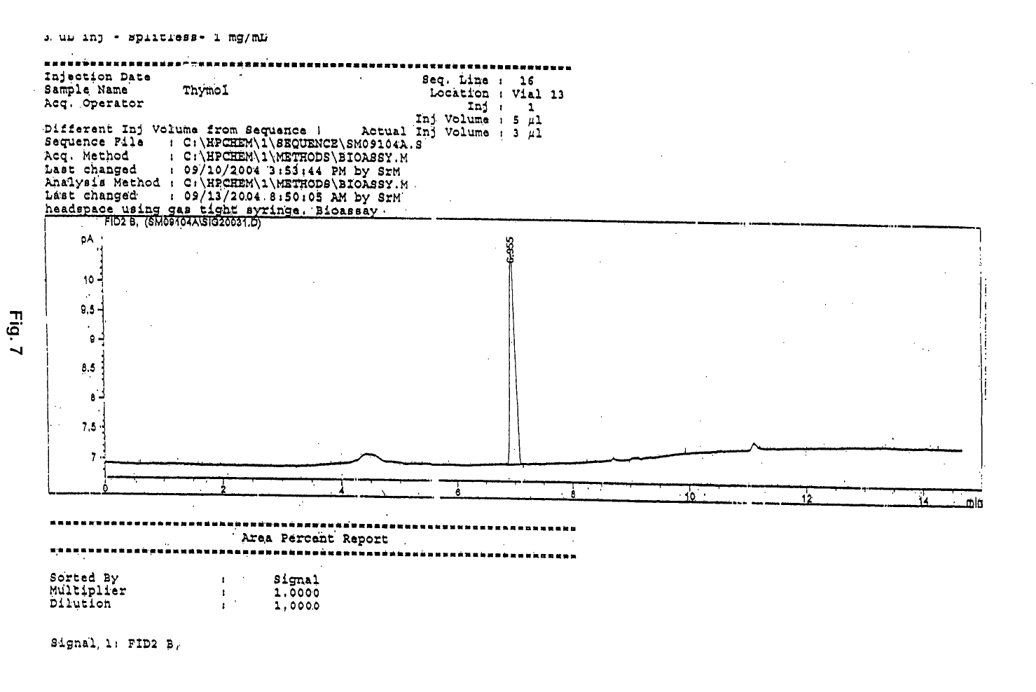

FIGS. 2-7 depict graphs from GC-FID analysis to determine volatility of the compounds BHT, cinnamyl alcohol, ferulic acid, eugenol, geraniol, and thymol.

### Example 4

### Scavenging Agents identified in DPPH and NORA Assays

Scavenging agents of the present invention, including several prospective volatile antioxidant compounds, were analyzed for their usefulness in a cosmetic or fragrance composition and/or methods of the present invention. The scavenging agents were first analyzed to determine their antioxidant activity against DPPH and nitric acid free radicals. The results are summarized below in Table 3.

TABLE 3
Antioxidant Activity of Scavenging Agents

| Scavenging Agent | DPPH EC₅₀¹ | NORA EC₅₀² |
|---|---|---|
| Raspberry Ketone | 12.05 | 10.14 |
| Jasmopyrane (Q) | NR³ | 150.44 |
| Paracymene | 22.22 | 274.16 |
| Lilial (G) | NR³ | 23.70 |
| Vanillin | 9.01 | 8.95 |
| LRG118 (RBD) | 52.63 | 131.17 |
| Florosa (Q) | NR³ | 150.33 |
| Orange Terpenes | 50.00 | 118.89 |
| Trolox (as reference) | 1.18 | 0.046 |
| Sodium Ascorbate (as reference) | 0.07 | — |
| Uric Acid (as reference) | — | 0.84 |

¹DPPH EC₅₀: Weight ratio of test material to react with a unit weight of DPPH, a free radical, by 50%.
²NORA EC₅₀: Nitric Oxide Radical Absorbance, the amount of test material (ug/mL) required to inhibit nitric oxide oxidation by 50% under assay conditions.
³NR: Not reportable. Insufficient inhibitory activity to give a result at a reasonable concentration.

As explained above, DPPH provides a measure of antioxidant activity against free radicals and NORA (Nitric Oxide Radical Absorbance) provides a measure of antioxidant activity against nitric oxide free radicals.

The data of Table 3 indicate that the compounds that contain aromatic phenols such as, Vanillin and Raspberry Ketone, have the greatest antioxidant capacity in both DPPH and nitric oxide free radical systems. Orange Terpenes seem to perform better as an inhibitor of nitric oxide free radicals. Lilial, an aromatic aldehyde, gave better than expected inhibitory response for nitric oxide, even though it does not contain a phenolic moiety. Jasmopyrane contains several oxygen atoms, but no aromatic moiety and performed more poorly than did lilial. The presence of an aromatic ring containing a free hydroxyl group plays a particularly important role in the antioxidant performance of these materials.

Compounds not possessing the aromatic/hydroxyl combination might still yield good antioxidant properties. For example, uric acid is a well-known nitric oxide scavenger and Retinol does not contain an aromatic moiety, yet functions as an antioxidant by combining with, and stabilizing, peroxyl free radicals.

Fragrance compounds that are generally identified as Balsamic (anise, balsam, caramel, chocolate, cinnamon, honey, sweet and vanilla) have structural traits, described herein, that indicate these compounds will perform well in a composition for scavenging reactive free radicals. Other materials such as resorcinol, catechol and parabens may also perform well.

Chemical structures of several tested scavenging agents are shown in Table 4.

TABLE 4
Chemical Structures of Various Scavenging Agents

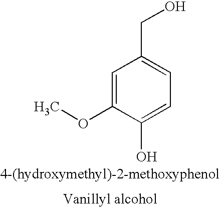

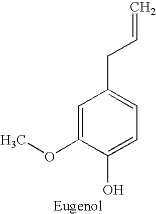

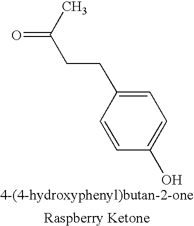

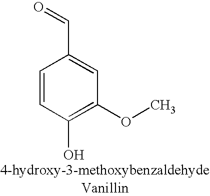

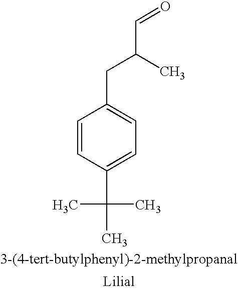

Additional analyses using the DPPH and NORA assays yielded results shown below in Table 5.

TABLE 5
Antioxidant Activity of Scavenging Agents

| | | | NORA |
|---|---|---|---|
| | | DPPH EC₅₀ | EC₅₀ |
| Compound Name | Fragrance | (g/g DPPH) | (ug/mL) |
| Vanillyl alcohol | Balsamic, sweet | 0.16 | 4.1 |
| Eugenol | Strong, spicy, clove | 0.12 | 0.8 |
| Ferulic acid | (Sour) | 0.19 | 0.3 |
| Thymol | Woody, burnt, smoky | 0.65 | >10.0 |
| Ethyl paraben | Medicinal (phenonip) | >2.82 | >10.0 |
| BHT | Faint musty, cresylic | 0.15 | >10.0 |
| Resorcinol | Odorless (medicinal) | 0.74 | >10.0 |
| m-Anisyl alcohol | Floral, mild, sweet | >2.82 | >10.0 |
| Cinnamyl alcohol | Sweet, balsamic, hyacinth | >2.82 | 11.8 |
| Maltol | Malt, toasted | 2.82 | 10.0 |
| 2-Phenoxyethanol | Medicinal (phenonip) | >2.82 | >10.0 |
| 3-Phenyl-1-propanol | Sweet, balsamic, hyacinth | >2.82 | >10.0 |
| Coumarin | New mown hay, sweet | >2.82 | >10.0 |
| | clover | | |
| Limonene | Mild, citrus, sweet, orange | >2.82 | >10.0 |
| Geraniol | Sweet, floral, rose, fruity | >2.82 | 1.9 |
| Camphor | Aromatic, woody, | >2.82 | >10.0 |
| | medicinal | | |
| Menthol | Minty, woody | >2.82 | >10.0 |
| Ethyl monanoate | Slight fatty, oily, nutty, | >2.82 | >10.0 |
| | fruity | | |

Eugenol and its metabolic precursor, ferulic acid, possessed excellent antioxidant properties. Vanillyl alcohol was also found to possess excellent antioxidant properties in the study resulting in Table 5. Thymol and resorcinol (used as a topical antiseptic) yielded good responses, while maltol performed marginally well.

Some of the chemical structures of the compounds discussed in Table 5 are depicted in Table 4. Thus, based on the results tabulated in Table 5 and a comparison of the structures in Table 4, scavenging agents of the present invention may have one or more of the following characteristics:

1. An aromatic ring;

2. At least one free hydroxyl group attached directly to the ring;

3. Absence of electron withdrawing groups attached to the ring (such as carboxylic acid/ester moieties);

4. Presence of one or more electron donating groups such as alkyl, ether, hydroxyl or amine groups attached directly to the ring;

5. At least one unsaturated hydrocarbon; and

6. Hydrocarbons containing at least one functional group selected from the group consisting of: carbonyl, amine, diamine, cyclic acetyl, and enol ether.

As explained above, these characteristics provide a stable molecule with rich electron densities that can rapidly scavenge oxidizing species such as nitric oxide free radicals and ozone.

### Example 5

### Determination of Antioxidant Property of a Topical Application for Reactivity with Nitrogen Dioxide/Ozone in Air (Artificial Skin Short Trough Method)

I. Purpose

This analysis may be conducted to determine the relative removal/reduction of nitrogen dioxide/ozone in air by passing an atmosphere elevated in nitrogen dioxide/ozone concentration through a reference and sample (scavenging agent test compound) cell containing sheets of artificial skin coated with a blank matrix (reference) or a matrix with active compound (sample). The difference in concentration of the pollutant air determined in the draw air from the two cells is an indication of chemical activity of the scavenging agent test compound with the challenge pollutant.

II. Scope

This method is applicable to any scavenging agent test compounds that may be used in practicing the present invention and includes all volatile raw materials or natural products capable of providing reactivity with nitrogen dioxide/ozone. The general method is applicable for any challenge atmosphere reactivity with a volatile scavenging agent (or substrate) held in the trough assembly. The volatile scavenging agent test compound should not adversely interact with the method of detection to cause interference. This method is generally used for a formulation or product applied to the skin using application techniques similar to those used for topical application of skin products.

III. Apparatus/Consumables

Ozone:

Ozone generator (small). Available from Fridge Guard™ (electronic antibacterial odor eliminator), Lucid Technology, Inc., 2102 West 7th Street Duluth, Minn. 55806, or equivalent.

Ozone Detector Tube, Gastec No. 18L, available from SKC, Inc 863 Valley View Road, Eighty-Four Pa., 15330.

D Cell Batteries (4)

Vac-U-Chamber (modified). Available from SKC, Inc., Fullerton, Calif., or equivalent. SKC, Inc 863 Valley View Road, Eighty-Four PA, 15330. Chamber has been modified to remove the front vacuum port and convert the sample port to a direct through-wall connection.

Nitrogen Dioxide:

10 ppm nitrogen dioxide in nitrogen. Compressed Gas. Scott Specialty Gases, Cylinder BAL4095, Project No. 08-29509, exp. date 200ct2006, Certified Working Class.

Nitrogen Oxides Detector Tube (NO+NO2), Gastec No. 11L, available from SKC, Inc 863 Valley View Road, Eighty-Four PA, 15330.

Plastic Bag—Clear Sample Handling Bags—Polyethylene.

Rubber Bands—Heavy

Tygon Tubing, ¼″ ID, 6-inch lengths

General:

Rubber stopper, single hole, #7 (two per analysis tube)

Glass/Polymer (rigid) tubing (small tube)—1.5 cm ID×20 cm with nipple end adapters

GasTec Pump Set GV-100S (set for 100 ml draw volume), available from SKC, Inc 863 Valley View Road, Eighty-Four PA, 15330.

Standard tubing and glassware.

Glove box—Model 818 GB or equivalent available from Plas Labs, Lansing Mich.

Vitro-Skin™—cut to 1 cm×20+ cm strips, available from IMS Testing Group, 282 Quarry Road, Milford, Conn. 06460-8508.

Fine mist spray bottle (2)

Polyethylene or polypropylene food container with tightly fitting lid capable of holding flat a 25 cm×30cm sheet.

Poly-mesh grid (rigid)—trim to 21×25 cm (4)

Aluminum support stand—3 or 4 layer, must fit in food container list in #Q.

Teflon short trough assembly©

1. Assembly consists of a solid lower Teflon block, an upper Teflon block with two inlet ports and two exhaust ports for a reference and sample trough milled into the face of the upper Teflon block.

2. The short trough utilizes a single U loop for each side (reference or sample), although other geometries using somewhat longer or shorter trough lengths are also possible.

3. A sheet of artificial skin with matrix coating is laid between the blocks with the coating facing the upper open trough block.

4. The combined sandwiched block is placed onto a block holder containing four clamp assemblies to firmly hold and press the blocks together. The prepared and coated artificial skin sheet acts as its own gasket for the assembly.

5. Air is drawn through the assembly over the coated artificial skin sheet from the glove box atmosphere, or gas bag, and though the color detector tube by means of the pump.

6. Trough depth is 3 mm and length is approximately 37 cm not including the inlet and outlet ports.

7. Inlet and outlet ports are solid Teflon and are specifically manufactured such that the outer diameter of the color detector tube is the inner diameter of the port.

8. The clamp base assembly was made of acetal block.

IV. Reagents/Solutions/Microorganisms

A. 10 ppm nitrogen dioxide in nitrogen. Compressed Gas. Scott Specialty Gases, Cylinder BAL4095, Project No. 08-29509, exp. date 200ct2006, Certified Working Class.

B. Raw material/solution/formulation containing volatile natural product.

C. Glycerin, reagent grade.

D. Propan-2-ol (isopropyl alcohol, IPA). Reagent grade

V. Instrument Parameters

A. The ozone generator is adapted with an electrical jumper to provide continuous generation.

B. An on/off switch was added between the batteries for the ozone generator.

C. The Gastec pump is set for 100 ml draw strokes.

VI. Procedure

Nitrogen dioxide:

A. Insert a 6-inch Tygon tube into a polyethylene bag leaving 1-2 inches of the tube sticking out of the bag.

B. Fold and/or roll the open edge of the bag around the tube leaving the open end of the tube in the bag unencumbered.

C. Stretch and wrap the rubber band around the tube and bag as many times as possible to secure the bag around the tube.

D. Inflate the bag with compressed air just to the point of applying slight pressure.

E. Manually dispense the air and crush the bag down from the closed.

F. Attach the bag to the nitrogen dioxide tank (with appropriate regulator) and slowly fill the bag to just less than fully expanded.

G. Attach a nipple to the bag and connect the nipple to a glass detector tube end (obtained from a used detector tube) or a glass tube equivalent. [This may be obtained by breaking a used detector tube end and polishing the broken end.] This is the gas bag. The gas bag is intended for immediate use.

H. Cover the open tube end with a plug and insert the bag into the glove box.

General:

I. Insert the clamp base assembly into the glove box.

J. Vitro-Skin Preparation

Humidification Chamber

a. Prepare a 30% glycerin by volume solution by adding 300 ml of glycerin to about 700 ml of DI water. Stir and mix well.

b. Add the 30% glycerin solution to the bottom of the food container.

c. Insert the aluminum support stand into the food container.

d. Close the food container.

Substrate Preparation

e. Cut the sheet to within 0.5 cm of the dimensions of the Teflon trough blocks

f. Prepare the sheet by laying the sheet on a clean glass or plastic surface uniformly and lightly sprayed with water from the fine mist sprayer. Textured side up.

g. Spray with DI water using a fine mist sprayer. Spray approximately every 2-3 cm up and down the sheet.

h. Using a dry balled-up wiper (similar to Kimwipes) wipe up and down the entire sheet.

i. Repeat Steps g, h.

j. Do not allow folds or bends in the sheet. The sheet will tend to curl along the edges.

k. Move the sheet onto the poly-mesh grid. Place the grid onto one of the levels of the aluminum support stand and seal the food container.

l. Allow the sheet to stand for a minimum of 2 hours in the food container.

m. Repeat steps e-l for each sheet to be utilized. [Typically, two food containers and up to eight levels are utilized at one time due to the preparation lead-time required.]

n. Add the topical application to the textured side of the sheet in very small amounts moving from the top of the sheet to the bottom, dragging the material along (spreading) in only one direction. Do not attempt to rub the topical application into the sheet in a circular manner as the sheet will very likely tear. Apply the reference topical application (no active) to the part of the sheet considered to align with the reference trough, and the sample topical application (with active) to the part of the sheet considered to align with the reference trough. It is advised to keep track of which side of the sheet contains the reference and the sample.

o. Lightly apply petrolatum to the surface of the Teflon open trough block making sure that all flat portions have a sufficient coating. A 3 or 5 cc syringe with a 16 gauge tip has been found to provide good lay-down of the petrolatum with practice.

p. Picking up one end of a sheet (short side) and lay the coated/textured side down on the Teflon open trough block. It is best to start at one end and slowly lower first the edge and then slowly let the sheet drape across the sheet to the final edge. Do not allow the sheet to move more than 1 or 2 mm once laid down.

q. Gently run a gloved finger over the flat surfaces to ensure a good adhesion to the flat area surrounding the troughs. It is advised to keep track of which side of the block contains the reference and the sample.

r. Flip the open trough over and carefully place the trough/sheet combination squarely on the flat surface of the other Teflon block.

s. Insert the combined block sandwich into the glove box and into the clamp base assembly.

t. Clamp. A light clamp pressure is sufficient. If no pressure is observed, the clamp will need to be adjusted to provide some pressure.

Ozone:

K. Place the ozone generator in the glove box with the on/off switch outside the glove box, seal the glove box, and allow the generator to operate continuously for 6 minutes, or until a steady concentration level required is attained.

L. Depending on the concentration desired and the steady-state ozone level in the glove box, either continue to operate the ozone generator or turn off the ozone generator.

General:

M. For ozone measurement leave the inlet end of the reference or sample open to the glove box; for nitrogen dioxide attached the gas bag to the reference or sample inlet port.

N. Break off the ends of the detector tube and insert the inlet side of the detector tube into the other end of the reference or sample port

O. Attach the outlet side of the detector tube to the pump. (An intermediate connector tube is acceptable.)

P. Pull the pump until locked using the 100 ml volume.

Q. The eye in pump plunger handle will turn white when sampling is complete.

R. Determine if the pollutant gas concentration (length of stain) is appropriate for the tube. If necessary use additional pump draws as indicated in the detector tube instructions. It is desirable to have the length of stain read between one-half and three quarters of the allowable stain range, if feasible.

S. Record the number of pump pulls and the detector tube length of stain reading.

T. Remove the detector tube.

U. Repeat the process for the sample using the same number of pump pulls as used for the reference. If no stain has been observed at this point, advance to the next lower sample range for the detector tube and the number of pump pulls for that sample range.

V. If no difference in length stain between reference and sample is still observed utilize a lower quantity/concentration of active material applied to the sample tube and repeat the entire testing sequence.

Ozone:

W. If the length of stain in the reference and sample tube is identical consider utilizing a short timed ozone generation period followed by detector tube measurement. Open the glove box between each reference and sample test to begin the timed sequence with fresh room air with each use. This timed sequence method is less precise and includes more inherent reproducibility error, but can produce effective results if a sufficient sample material activity is present.

Nitrogen dioxide:

X. If the length of stain in the reference and sample tube is identical consider reducing the concentration of the gas in the bag. To do this partially pre-inflate the flat/crushed bag with compressed air in the dilution ratio required then fill the remainder of the bag from the compressed nitrogen dioxide tank. This dilution method is less precise and includes more inherent reproducibility error, but can produce effective results if a sufficient sample material activity is present. [Immediately following sampling, plug the bag closed. The bag atmosphere may continue to be used for subsequent tests for several minutes without appreciable change in the nitrogen dioxide concentration.]

Y. At the completion of testing, clean the Teflon rough blocks with propan-2-ol and laboratory wipers. Clean the blocks as soon as possible after testing and allow to air dry in a protected location.

VIII. Results and Calculations

Results

1. Determine the concentration of nitrogen dioxide/ozone from the chart range included with the detector tubes. The concentration is a combination of the length of stain reading on the tube and the number of pump pulls.

2. Evidence of antioxidant activity is a lower concentration of nitrogen dioxide/ozone in the detector tube connected to the sample.

3. An approximate reduction percentage can be determined if desired.

Calculations

$$\% reduction = ( \frac{Concentration [ reference ] - Concentration [ sample ]}{Concentration [ reference ]} ) \times 100$$

Using the teflon short trough block a scavenging agent test compound comprising lemon grass oil dispersed as a liquid in a semi-solid material matrix and a scavenging test compound comprising a vanillal bourbonal powder dissolved in an alcohol and applied over the semi-solid matrix were shown to reduce ozone approximately 88% and nitrogen dioxide approximately 13%, respectively.

In particular, following the above-described procedure, 5 drops of lemon oil grass was applied as an active compound in 20 grams of petrolatum, which constitutes 0.09g in 20.09 grams or 0.45% of the formulation. Application of the lemon grass oil test compound was about 0.0025 g/cm². Application of the lemon grass oil at this concentration resulted in average reductions of ozone in amounts of 85%, 88%, 88%, 89%, 90% and 90% for an average reduction of approximately 88%.

In addition, following the above-described procedure, vanillal bourbonal was applied as a solution of 2.12 g/50g of propan-2-ol. With 10 sprays per 129 cm² area (8 inches×2.5 inches), and a presumption that at best 50% of the spray adheres, vanillal bourbonal was applied at a concentration of approximately 0.000164 g/cm². This concentration of application roughly corresponds to an active scavenging agent comprising approximately 6.5% of an aerosol formulation of a composition of the present invention. Application of the vanillal bourbonal at this concentration resulted in average reductions of nitric dioxides (as nitric oxides) in amounts of 7%, 9%, 9%, 13%, 20%, and 22% for an average reduction of approximately 13%. Reduction of nitric oxides was seen at low concentrations of nitric oxides and when contact surface was high.

### Example 6

### Determination of Antioxidant Property of a Topical Application for Reactivity with Nitrogen Dioxide/Ozone in Air (Artificial Skin Long Trough Method)

The Artificial Skin Trough Method described above in Example 5 may also be conducted using a long trough rather than the short trough described above. In particular, rather than the Teflon short trough assembly described above, the following Teflon long trough assembly may be followed:

A. Teflon long trough assembly©.

1. Assembly consists of a solid lower Teflon block, an upper Teflon block with two inlet ports and two exhaust ports for a reference and sample trough milled into the face of the upper Teflon block.

2. The long trough utilizes a snake-like S loop for each side (reference or sample), although other geometries using somewhat longer or shorter trough lengths are also possible.

3. A sheet of artificial skin with matrix coating is laid between the blocks with the coating facing the upper open trough block.

4. The combined sandwiched block is placed onto a block holder containing four clamp assemblies to firmly hold and press the blocks together. The prepared and coated artificial skin sheet acts as its own gasket for the assembly.

5. Air is drawn through the assembly over the coated artificial skin sheet from the glove box atmosphere, or gas bag, and though the color detector tube by means of the pump.

6. Trough depth is 3 mm and length is approximately 132 cm not including the inlet and outlet ports.

7. Inlet and outlet ports are solid Teflon and are specifically manufactured such that the outer diameter of the color detector tube is the inner diameter of the port.

8. The clamp base assembly was made of acetal block.

One of ordinary skill in the art will appreciate that the long trough method allows for a larger contact surface area to be tested. Therefore, test results using the long trough assembly are expected to show greater reductions in nitrogen oxides using a scavenging agent of the present invention.

### Example 7

### Air Fresheners/Deodorants

Air freshener gels may be used in homes, buildings and cars. Similarly, the air freshener gel may be applied in aerosol form as a personal deodorant for all over body use. These applications comprise the general formula:

| | |
|---|---|
| purified water | 86.55% |
| butylene glycol | 10.00 |
| Gellan gum | 1.00 |
| sodium chloride | 0.25 |
| Polysorbate-20 | 1.00 |
| fragrance component/scavenging agent: | 1.00 |
| diazolidinyl urea (and) iodopropynyl | 0.20 |
| butylcarbamate | |

Air freshener spray pumps have the general formula 98% SD alcohol and 2% fragrance component. In practicing the present invention, the fragrance component may include a scavenging agent.

Air freshener aerosols have the general formula:

| | |
|---|---|
| SD alcohol | 68.60% |
| fragrance component/scavenging agent | 1.40 |
| propane (propellant) | 12.96 |
| isobutane (propellant) | 17.04 |

Personal colognes have the general formula 92.50% SD alcohol, 5.00% fragrance component, which may include a scavenging agent, and 2.50% purified water.

### Example 8

### Air Treatment Systems

Air treatment systems that include anti-bacterial or deordorant functions may use a gel or aerosol of the general formula listed in Example 1. The filter of the air treatment system is dipped, treated with, or has sprayed onto it the fragrance components, which may include a scavenging agent, wherein the fragrance components have volatile and antioxidant properties as described herein.

### Example 9

### Candles

Stick candles may be used to release the scavenging agent, such as a volatile antioxidant into the air surrounding the candle, where the agent will scavenge reactive oxygen and nitrogen species in the air. Gel candles have been described in U.S. Pat. No. 5,879,694, incorporated herein by reference, or the candles have the general formula:

| | |
|---|---|
| paraffin wax | 97.495% |
| stearamide | 1.00 |
| dye | 0.005 |
| fragrance component/scavenging agent | 1.00 |
| polyetheylene or stearic acid | 0.50 |

### Example 10

### Carpet Fresheners

Carpet fresheners as antioxidants have the general formula 98% sodium bicarbonate and 2% fragrance component. In practicing the present invention, the fragrance component may include a scavenging agent.

### Example 11

### Moisturizer/Cosmetic Foundations

A volatile scavenging agent may be combined with a vehicle to produce a cosmetic or fragrance product. For example, as discussed above, a composition according to the present invention may comprise a combination of a scavenging agent and a vehicle such as a pharmaceutically or cosmetically acceptable vehicle or carrier. Also as discussed above, the compositions of the present invention may be formulated into products such as a solution, gel, lotion, cream, ointment, oil-in-water emulsion, water-in-oil emulsion, or other pharmaceutically or cosmetically acceptable form. The complex compositions of the present invention may also contain various known and conventional cosmetic ingredients so long as they do not detrimentally affect the desired effects.

In one embodiment, a user of a composition of the present invention topically applies a moisturizer or cosmetic foundation to an area of skin. The scavenging agent in the composition volatilizes into a volume of air adjacent to the skin where the product was applied and provides protection against free radicals, including reactive oxygen or nitrogen species or precursors to their radicals.

Generally moisturizer formulations comprise:

| | |
|---|---|
| Purified water | 78.30% |
| Disodium EDTA | 0.15 |
| Glycereth-26 | 3.00 |
| Capric/caprylic triglycerides | 5.00 |
| Isononyl isononaoate | 5.00 |
| Petrolatum | 3.00 |
| Polyglyceryl-2 triisostearate | 1.79 |
| Polysorbate 60 | 1.71 |
| Polyacrylamide (and) C13-14 Isoparaffin (and) | 1.75 |
| Laureth-7 | |
| Fragrance component/scavenging agent | 0.10 |
| Diazolidinyl urea and iodopropynyl butyl carbamate | 0.20 |

Generally, cosmetic foundations include:

| | |
|---|---|
| Cyclopentasiloxane | 17.00% |
| Cyclomethicone | 8.00 |
| Cetyl PEG/PPG - 10/1 Dimethicone (and) | 3.00 |
| Polyglyceryl-4 Isostearate (and) Hexyl Laurate | |
| Cetyl PEG/PPG - 10/1 Dimethicone | 2.00 |
| Cetyl Dimethicone | 4.00 |
| Octyldodecyl Neopentanoate | 4.00 |
| Cetearyl Ethylhexanoate | 5.00 |
| Cyclomethicone (and) Quaternium-18 | |
| Hectorite (and) SD alcohol 40 | 6.00 |
| Titanium Dioxide (and) (Triethoxy)Caprylylsilane | 6.78 |
| Yellow Iron Oxide (and) Caprylylsilane | 0.74 |
| Red Iron Oxide (and) Caprylylsilane | 0.32 |
| Black Iron Oxide (and) Caprylylsilane | 0.16 |
| Talc (and) Methicone, Hydrophobic | 3.00 |
| Ethylene/Acrylic Acid Copolymer | 4.00 |
| Purified water | 30.40 |
| Tocopheryl acetate | 0.30 |
| Glycereth-26 | 4.00 |
| Diazolidinyl urea (and) iodopropynyl butylcarbamate | 0.30 |
| Sodium chloride | 0.50 |
| Fragrance component/scavenging agent | 0.10 |

1. A method of scavenging a reactive molecule in a volume of air surrounding, near, or adjacent to a site of application of a composition comprising releasing into the volume of air a composition comprising a scavenging agent that comprises a fragrance component, wherein the fragrance component includes at least one of: (i) an aromatic ring; (ii) a free hydroxyl group attached directly to an aromatic ring; (iii) an aromatic ring that does not have any electron withdrawing group attached to the ring; (iv) an electron donating group selected from the group consisting of alkyl, ether, hydroxyl, amine and combinations thereof attached to an aromatic ring, (v) at least one unsaturated hydrocarbon; and (vi) hydrocarbons containing at least one functional group selected from the group consisting of: carbonyl, amine, diamine, cyclic acetyl, and enol ether, wherein the reactive molecule is selected from the group consisting of a free radical, a reactive oxygen species, and a reactive nitrogen species.

2. The method of claim 1, wherein the scavenging agent is volatile at a temperature ranging from approximately 5-45° C.

3. The method of claim 2, wherein the scavenging agent further comprises a non-volatile antioxidant.

4. The method of claim 3, further comprising applying the scavenging agent to an area of skin, wherein the scavenging agent further comprises a cosmetically acceptable vehicle.

5. The method of claim 1, wherein the step of releasing the scavenging agent into the volume of air further comprises a first step selected from the group consisting of: the step of first lighting a candle; the step of first scratching a pet litter; and the step of first activating a deodorizer.

6. The method of claim 1, wherein the reactive oxygen species is selected from the group consisting of: superoxide (O₂⁻), ozone (O₃), hydrogen peroxide (H₂O₂), peroxy radicals (HO₂ and RO₂), alkyl peroxide (R₂O₂), hydroxyl radical (OH), alkoxy radical (OR) and singlet oxygen.

7. The method of claim 1, wherein the reactive nitrogen species is a nitric oxide.

8. The method of claim 1, wherein the scavenging agent is selected from the group consisting of: vanillin, vanillyl alcohol, vanillal bourbonal, raspberry ketone, eugenol, ferulic acid, orange terpenes, lilial, lemon grass oil, and combinations thereof.

9. The method of claim 8, wherein the scavenging agent is lemon grass oil and further wherein the reactive molecule is ozone.

10. The method of claim 8, wherein the scavenging agent is vanillal bourbonal and further wherein the reactive molecule is a nitric oxide.

11. The method of claim 8, wherein the step of releasing a scavenging agent into the volume of air further comprises the steps of first mixing the scavenging agent with a vehicle to form a mixture and applying the mixture to an area of skin.

12. The method of claim 11, wherein the vehicle includes a moisturizer.

13. The method of claim 8, wherein the step of releasing the scavenging agent into the volume of air further comprises a first step selected from the group consisting of: the step of first lighting a candle; the step of first scratching a pet litter; and the step of first activating a deodorizer.

14. A composition for scavenging a reactive molecule in a volume of air surrounding, near, or adjacent to a site of application of the composition comprising approximately 0.01-20% by weight a scavenging agent, wherein the scavenging agent is volatile at a temperature ranging from 5-45° C. and comprises a fragrance component, wherein the fragrance component includes at least one of: (i) an aromatic ring, (ii) a free hydroxyl group attached directly to an aromatic ring; (iii) an aromatic ring that does not have any electron withdrawing group attached to the ring; (iv) an electron donating group selected from the group consisting of alkyl, ether, hydroxyl, amine and combinations thereof attached to an aromatic ring, (v) at least one unsaturated hydrocarbon; and (vi) hydrocarbons containing at least one functional group selected from the group consisting of: carbonyl, amine, diamine, cyclic acetyl, and enol ether, wherein the composition scavenges reactive molecules selected from the group consisting of free radicals, reactive oxygen species, and reactive nitrogen species.

15. The composition of claim 14, further comprising a non-volatile scavenging agent.

16. The composition of claim 14, wherein the scavenging agent is selected from the group consisting of vanillin, vanillyl alcohol, vanillal bourbonal, raspberry ketone, eugenol, ferulic acid, orange terpenes, lilial, lemon grass oil, and combinations thereof

17. The composition of claim 14, further comprising a cosmetically acceptable vehicle.

18. The composition of claim 17, wherein the cosmetically acceptable vehicle is selected from the group consisting of a moisturizing product and a cosmetic foundation.

19. The composition of claim 18, wherein the scavenging agent is lemon grass oil and the reactive molecule is ozone.

20. The composition of claim 18, wherein the scavenging agent is vanillal bourbonal and the reactive molecule is a nitric oxide.

21. A composition for scavenging reactive molecules in a volume of air surrounding, near, or adjacent to a site of application of the composition consisting essentially of a cosmetically acceptable carrier and approximately 0.01-20% by weight of the total composition a scavenging agent, wherein the scavenging agent is volatile at a temperature ranging from 5-45° C. and comprises a fragrance component, wherein the fragrance component includes at least one of: (i) an aromatic ring, (ii) a free hydroxyl group attached directly to an aromatic ring; (iii) an aromatic ring that does not have any electron withdrawing group attached to the ring; (iv) an electron donating group selected from the group consisting of alkyl, ether, hydroxyl, amine and combinations thereof attached to an aromatic ring (v) at least one unsaturated hydrocarbon; and (vi) hydrocarbons containing at least one functional group selected from the group consisting of: carbonyl, amine, diamine, cyclic acetyl, and enol ether, wherein the composition scavenges reactive molecules selected from the group consisting of free radicals, reactive oxygen species, and reactive nitrogen species.
